SEQUENCE LISTING (60 sequences)

SEQ ID NO: 1 - protein, 350 aa, synthetic construct
  REGION 1..350: note = Mitogenic T-cell activating transmembrane protein (OKT3-CD8STK-TM-A)
METDTLLLWVLLLWVPGSTGQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSSGGGGSGGGGSGGGGSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRSDPTTTPAPRPPTPAPTIASQPLSLRPEACRPAAGGAVHTRGLDFACDIYIWAPLAGTCGVLLLSLVITLYCNHRNRRRVCKCPRPVV

SEQ ID NO: 2 - protein, 358 aa, synthetic construct
  REGION 1..358: note = Mitogenic T-cell activating transmembrane protein (15E8-CD8STK-TM-A)
METDTLILWVLLLLVPGSTGQVQLKESGPGLVAPSQSLSITCTVSGFSLTSYGVHWVRQPPGKGLEWLGVIWAGGSTNYNSALMSRLSISKDNSKSQVFLKMNSLQTDDTAMYYCARDKRAPGKLYYGYPDYWGQGTTLTVSSGGGGSGGGGSGGGGSDIVLTQSPASLAVSLGQRATISCRASESVEYYVTSLMQWYQQKPGQPPKLLIYAASNVESGVPARFSGSGSGTDFSLNIHPVEEDDIAMYFCQQTRKVPSTFGGGTKLEIKRSDPTTTPAPRPPTPAPTIASQPLSLRPEACRPAAGGAVHTRGLDFACDIYIWAPLAGTCGVLLLSLVITLYCNHRNRRRVCKCPRPVV

SEQ ID NO: 3 - protein, 351 aa, synthetic construct
  REGION 1..351: note = Mitogenic T-cell activating transmembrane protein (TGN1412-CD8STK-TM-A)
METDTLILWVLLLLVPGSTGQVQLVQSGAEVKKPGASVKVSCKASGYTFTSYYIHWVRQAPGQGLEWIGCIYPGNVNTNYNEKFKDRATLTVDTSISTAYMELSRLRSDDTAVYFCTRSHYGLDWNFDVWGQGTTVTVSSGGGGSGGGGSGGGGSDIQMTQSPSSLSASVGDRVTITCHASQNIYVWLNWYQQKPGKAPKLLIYKASNLHTGVPSRFSGSGSGTDFTLTISSLQPEDFATYYCQQGQTYPYTFGGGTKVEIKRSDPTTTPAPRPPTPAPTIASQPLSLRPEACRPAAGGAVHTRGLDFACDIYIWAPLAGTCGVLLLSLVITLYCNHRNRRRVCKCPRPVV

SEQ ID NO: 4 - protein, 7 aa, synthetic construct
  REGION 1..7: note = OKT3 CDRH1
GYTFTRY

SEQ ID NO: 5 - protein, 6 aa, synthetic construct
  REGION 1..6: note = OKT3 CDRH2
NPSRGY

SEQ ID NO: 6 - protein, 10 aa, synthetic construct
  REGION 1..10: note = OKT3 CDRH3
YYDDHYCLDY

SEQ ID NO: 7 - protein, 10 aa, synthetic construct
  REGION 1..10: note = OKT3 CDRL1
SASSSVSYMN

SEQ ID NO: 8 - protein, 7 aa, synthetic construct
  REGION 1..7: note = OKT3 CDRL2
DTSKLAS

SEQ ID NO: 9 - protein, 9 aa, synthetic construct
  REGION 1..9: note = OKT3 CDRL3
QQWSSNPFT

SEQ ID NO: 10 - protein, 7 aa, synthetic construct
  REGION 1..7: note = 15E8 CDRH1
GFSLTSY

SEQ ID NO: 11 - protein, 5 aa, synthetic construct
  REGION 1..5: note = 15E8 CDRH2
WAGGS

SEQ ID NO: 12 - protein, 15 aa, synthetic construct
  REGION 1..15: note = 15E8 CDRH3
DKRAPGKLYYGYPDY

SEQ ID NO: 13 - protein, 15 aa, synthetic construct
  REGION 1..15: note = 15E8 CDRL1
RASESVEYYVTSLMQ

SEQ ID NO: 14 - protein, 7 aa, synthetic construct
  REGION 1..7: note = 15E8 CDRL2
AASNVES

SEQ ID NO: 15 - protein, 9 aa, synthetic construct
  REGION 1..9: note = 15E8 CDRL3
QQTRKVPST

SEQ ID NO: 16 - protein, 6 aa, synthetic construct
  REGION 1..6: note = TGN1412 CDRH1
GYTFSY

SEQ ID NO: 17 - protein, 6 aa, synthetic construct
  REGION 1..6: note = TGN1412 CDRH2
YPGNVN

SEQ ID NO: 18 - protein, 11 aa, synthetic construct
  REGION 1..11: note = TGN1412 CDRH3
SHYGLDWNFDV

SEQ ID NO: 19 - protein, 10 aa, synthetic construct
  REGION 1..10: note = TGN1412 CDRL1
HASQNIYVLN

SEQ ID NO: 20 - protein, 7 aa, synthetic construct
  REGION 1..7: note = TGN1412 CDRL2
KASNLHT

SEQ ID NO: 21 - protein, 9 aa, synthetic construct
  REGION 1..9: note = TGN1412 CDRL3
QQGQTYPYT

SEQ ID NO: 22 - protein, 173 aa, synthetic construct
  REGION 1..173: note = OX40L sequence
MERVQPLEENVGNAARPRFERNKLLLVASVIQGLGLLLCFTYICLHFSALQVSHRYPRIQSIKVQFTEYKKEKGFILTSQKEDEIMKVQNYLISLKGYFSQEVNISLHYQKDEEPLFQLKKVRSVNSLMVASLTYKDKVYLNVTTDNTSLDDFHVNGGELILIHQNPGEFCVL

SEQ ID NO: 23 - protein, 254 aa, synthetic construct
  REGION 1..254: note = 41BBL sequence
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 24 - protein, 234 aa, synthetic construct
  REGION 1..234: note = spacer sequence, hinge-CH2CH3 of human IgG1
AEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKD

SEQ ID NO: 25 - protein, 46 aa, synthetic construct
  REGION 1..46: note = spacer sequence, human CD8 stalk
TTTPAPRPPTPAPTIASQPLSLRPEACRPAAGGAVHTRGLDFACDI

SEQ ID NO: 26 - protein, 20 aa, synthetic construct
  REGION 1..20: note = spacer sequence, human IgG1 hinge
AEPKSPDKTHTCPPCPKDPK

SEQ ID NO: 27 - protein, 185 aa, synthetic construct
  REGION 1..185: note = spacer sequence, CD2 ectodomain
KEITNALETWGALGQDINLDIPSFQMSDDIDDIKWEKTSDKKKIAQFRKEKETFKEKDTYKLFKNGTLKIKHLKTDDQDIYKVSIYDTKGKNVLEKIFDLKIQERVSKPKISWTCINTTLTCEVMNGTDPELNLYQDGKHLKLSQRVITHKWTTSLSAKFKCTAGNKVSKESSVEPVSCPEKGLD

SEQ ID NO: 28 - protein, 259 aa, synthetic construct
  REGION 1..259: note = spacer sequence, CD34 ectodomain
SLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKT

SEQ ID NO: 29 - protein, 153 aa, synthetic construct
  REGION 1..153: note = IL2 sequence
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 30 - protein, 177 aa, synthetic construct
  REGION 1..177: note = IL7 sequence
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 31 - protein, 162 aa, synthetic construct
  REGION 1..162: note = IL15 sequence
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 32 - protein, 271 aa, synthetic construct
  REGION 1..271: note = cytokine-based T-cell activating transmembrane protein sequence
MAHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHSGGGSPAKPTTTPAPRPPTPAPTIASQPLSLRPEACRPAAGGAVHTRGLDFACDIYIWAPLAGTCGVLLLSLVITLYCNHRNRRRVCKCPRPVV

SEQ ID NO: 33 - protein, 256 aa, synthetic construct
  REGION 1..256: note = cytokine-based T-cell activating transmembrane protein sequence
MGLVRRGARAGPRMPRGWTALCLLSLLPSGFMAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSPAKPTTTPAPRPPTPAPTIASQPLSLRPEACRPAAGGAVHTRGLDFACDIYIWAPLAGTCGVLLLSLVITLYCNHRNRRRVCKCPRPVV

SEQ ID NO: 34 - protein, 20 aa, synthetic construct
  REGION 1..20: note = self-cleaving site
RAEGRGSLLTCGDVEENPGP

SEQ ID NO: 35 - protein, 20 aa, synthetic construct
  REGION 1..20: note = self-cleaving site
QCTNYALLKLAGDVESNPGP

SEQ ID NO: 36 - protein, 8 aa, synthetic construct
  REGION 1..8: note = biotin mimic, streptagII
WSHPQFEK

SEQ ID NO: 37 - protein, 15 aa, synthetic construct
  REGION 1..15: note = biotin mimic, flankedccstreptag
AECHPQGPPCIEGRK

SEQ ID NO: 38 - protein, 8 aa, synthetic construct
  REGION 1..8: note = biotin mimic, ccstreptag
CHPQGPPC

SEQ ID NO: 39 - protein, 15 aa, synthetic construct
  REGION 1..15: note = biotin mimic, Long nanotag
DVEAWLDERVPLVET

SEQ ID NO: 40 - protein, 9 aa, synthetic construct
  REGION 1..9: note = biotin mimic, Short nanotag
DVEAWLGAR

SEQ ID NO: 41 - protein, 8 aa, synthetic construct
  REGION 1..8: note = biotin mimic, Streptag
WRHPQFGG

SEQ ID NO: 42 - protein, 38 aa, synthetic construct
  REGION 1..38: note = biotin mimic, SBP-tag
MDEKTTGWRGGHVVEGLAGELEQLRARLEHHPQGQREP

SEQ ID NO: 43 - protein, 36 aa, synthetic construct
  REGION 1..36: note = binding domain, StreptagII-d8-x2
WSHPQFEKSGGGGSPAPRPPTPAPTIASWSHPQFEK

SEQ ID NO: 44 - protein, 50 aa, synthetic construct
  REGION 1..50: note = binding domain, Flankedccstreptag-d8-x2
ECHPQGPPCIEGRKSSGGGGSPAPRPPTPAPTIASECHPQGPPCIEGRKS

SEQ ID NO: 45 - protein, 251 aa, synthetic construct
  REGION 1..251: note = glutathione S-transferase (GST) domain
MGTSLLCWMALCLLGADHADAMSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLEVLFQGPLG

SEQ ID NO: 46 - protein, 17 aa, synthetic construct
  REGION 1..17: note = Rituximab-binding epitope sequence from CD20
CEPANPSEKNSPSTQYC

SEQ ID NO: 47 - protein, 10 aa, synthetic construct
  REGION 1..10: note = cysteine-constrained 7-mer cyclic peptide, R15-C
ACPYANPSLC

SEQ ID NO: 48 - protein, 10 aa, synthetic construct
  REGION 1..10: note = cysteine-constrained 7-mer cyclic peptide, R3-C
ACPYSNPSLC

SEQ ID NO: 49 - protein, 10 aa, synthetic construct
  REGION 1..10: note = cysteine-constrained 7-mer cyclic peptide, R7-C
ACPFANPSTC

SEQ ID NO: 50 - protein, 10 aa, synthetic construct
  REGION 1..10: note = cysteine-constrained 7-mer cyclic peptide, R8-, R12-, R18-C
ACNFSNPSLC

SEQ ID NO: 51 - protein, 10 aa, synthetic construct
  REGION 1..10: note = cysteine-constrained 7-mer cyclic peptide, R14-C
ACPFSNPSMC

SEQ ID NO: 52 - protein, 10 aa, synthetic construct
  REGION 1..10: note = cysteine-constrained 7-mer cyclic peptide, R16-C
ACSWANPSQC

SEQ ID NO: 53 - protein, 10 aa, synthetic construct
  REGION 1..10: note = cysteine-constrained 7-mer cyclic peptide, R17-C
ACMFSNPSLC

SEQ ID NO: 54 - protein, 10 aa, synthetic construct
  REGION 1..10: note = cysteine-constrained 7-mer cyclic peptide, R19-C
ACPFANPSMC

SEQ ID NO: 55 - protein, 10 aa, synthetic construct
  REGION 1..10: note = cysteine-constrained 7-mer cyclic peptide, R2-C
ACWASNPSLC

SEQ ID NO: 56 - protein, 10 aa, synthetic construct
  REGION 1..10: note = cysteine-constrained 7-mer cyclic peptide, R10-C
ACEHSNPSLC

SEQ ID NO: 57 - protein, 10 aa, synthetic construct
  REGION 1..10: note = cysteine-constrained 7-mer cyclic peptide, R13-C
ACWAANPSMC

SEQ ID NO: 58 - protein, 12 aa, synthetic construct
  REGION 1..12: note = mimetope of Rituximab
QDKLTQWPKWLE

SEQ ID NO: 59 - protein, 16 aa, synthetic construct
  REGION 1..16: note = QBEnd10-binding epitope from within the CD34 antigen
ELPTQGTFSNVSTNVS

SEQ ID NO: 60 - protein, 136 aa, synthetic construct
  REGION 1..136: note = binding domain RQR8
CPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVV

(19) United States
(12) Patent Application Publication
(10) Pub. No.: US 2025/0388929 A1
(43) Pub. Date: Dec. 25, 2025

(54) Retroviral and Lentiviral Vectors

(71) Applicant: AUTOLUS LIMITED, London (GB)

(72) Inventors: Martin Pulé, London (GB); Leila Mekkaoui, London (GB)

(21) Appl. No.: 18/987,004

(22) Filed: Dec. 19, 2024

Related U.S. Application Data
(63) Continuation of application No. 18/806,375, filed on Aug. 15, 2024, now abandoned.
Application No. 18/806,375 is a continuation of application No. 18/601,311, filed on Mar. 11, 2024, now abandoned.
Application No. 18/601,311 is a continuation of application No. 18/471,846, filed on Sep. 21, 2023, now abandoned.
Application No. 18/471,846 is a continuation of application No. 17/192,561, filed on Mar. 4, 2021, now Pat. No. 11,814,641.
Application No. 17/192,561 is a continuation of application No. 15/554,499, filed on Aug. 30, 2017, now Pat. No. 10,954,530.

(30) Foreign Application Priority Data
Mar. 2, 2015 (GB) 1503500.9

(51) Int. Cl. C12N 15/86; A61K 35/76; A61K 38/00; A61K 39/00; C07K 14/00; C07K 14/725; C07K 16/00; C07K 16/28

(52) U.S. Cl. CPC C12N 15/86; A61K 35/76; A61K 38/00; C07K 14/00; C07K 14/7051; C07K 16/00; C07K 16/2809; C07K 16/2818; A61K 2039/507; C07K 2317/622; C07K 2317/75; C12N 2740/13032; C12N 2740/13045; C12N 2740/13052; C12N 2740/15032; C12N 2740/15045; C12N 2740/15052; C12N 2810/852; C12N 2810/855; C12N 2810/859

(57) ABSTRACT

The present invention provides a retroviral or lentiviral vector having a viral envelope which comprises: (i) a mitogenic T-cell activating transmembrane protein which comprises a mitogenic domain and a transmembrane domain; and/or (ii) a cytokine-based T-cell activating transmembrane protein which comprises a cytokine domain and a transmembrane domain, wherein the mitogenic or cytokine-based T-cell activating transmembrane protein is not part of a viral envelope glycoprotein. When cells such as T-cells of Natural Killer cells are transduced by such a viral vector, they are simultaneously activated by the mitogenic T-cell activating transmembrane protein and/or the cytokine-based T-cell activating transmembrane protein.

### CROSS-REFERENCE TO RELATED APPLICATIONS

This is a Continuation of U.S. application Ser. No. 18/806,375, filed Aug. 15, 2024, now abandoned, which is a Continuation of U.S. application Ser. No. 18/601,311, filed Mar. 11, 2024, now abandoned, which is a Continuation of U.S. application Ser. No. 18/471,846, filed Sep. 21, 2023, now abandoned, which is a Continuation of U.S. application Ser. No. 17/192,561, filed Mar. 4, 2021, now U.S. Pat. No. 11,814,641, issued Nov. 14, 2023, which is a Continuation of U.S. application Ser. No. 15/554,499, filed Aug. 30, 2017, now U.S. Pat. No. 10,954,530, issued Mar. 23, 2021, which is a U.S. National Phase of International Application No. PCT/GB2016/050537, filed Mar. 1, 2016, which claims priority to Great Britain Application No. 1503500.9, filed Mar. 2, 2015.

### INCORPORATION BY REFERENCE OF THE SEQUENCE LISTING

This application contains, as a separate part of disclosure, a Sequence Listing in computer-readable form (Filename: 52305E_SeqListing.xml; 79,840 bytes-XML file dated Sep. 19, 2023) which is incorporated by reference herein in its entirety.

### FIELD OF THE INVENTION

The present invention relates to retroviral and lentiviral vectors and cells for their production. The vectors may be used for transducing cells, such as T-cells. In particular, the invention relates retroviral or lentiviral vectors capable of both transducing and activating a cell, such as a T cell.

### BACKGROUND TO THE INVENTION

The generation of engineered T-cell products typically requires stimulation with a mitogen followed by transduction with an integrating vector, such as a lentiviral vector or a retroviral vector.

A widely used approach is to add soluble mitogenic monoclonal antibodies (mAb), such as anti-TCR/CD3 and anti-CD28, to the cell culture. An alternative approach is to attach anti-TCR/CD3 mAb along with anti-CD28 mAb to a bead. The surface of the bead has improved T cell activating properties compared to the soluble antibodies alone.

In addition cytokines (e.g. IL2, IL15 or IL7) are commonly added to the cell culture.

These mitogen antibodies and cytokines are single-use consumables and typically represent the most costly part of the T-cell production process.

Maurice et al. describe the direct engineering of a lentiviral envelope protein such that the CD3 agonist OKT3 is displayed on the virion surface (Maurice et al.; Blood; 2002; 99; 2342-2350). Verhoeyen et al. describe a similar approach in which the lentiviral envelope protein is engineered to incorporate IL7 (Verhoeyen et al.; Blood; 2003; 101; 2167-2174).

Each of these engineering approaches requires complex engineering of the viral envelope protein. This complex engineering must be performed for each discrete peptide to be displayed on the virion surface. The approach has also been shown to reduce viral titre.

There is thus a need for new approaches for generating engineered T cell products which are not associated with the disadvantages described above.

### DESCRIPTION OF THE FIGURES

FIG. 1—Diagram of a retroSTIM vector surrounded by a lipid bilayer which is studded with the RD114 envelope glycoprotein and various mitogenic elements such as scFv or membrane-bound cytokines.

FIG. 2—Demonstration that an OKT3 scFv can be incorporated into a lentivirus. Results show activation of T cells (a) non-stimulated-transduced with lentiviral vector from 293T cells; (b) stimulated with OKT3, CD28.2 and IL2-transduced with lentiviral vector from 293T cells; (c) non-stimulated-transduced with supernatant from 293T.OKT3, transfected with only the transfer vector; (d) non-stimulated-transduced with lentiviral vector from 293T.OKT3. Top panel shows scatter-plots of transduction (x-axis), and activation by CD25 expression (y-axis). Bottom panel show photomicrographs of T-cell cultures. Clumping indicated activation.

FIG. 3—Demonstration that mitogenic stimulation and transduction of T cells is dependent on gagpol. 293T cells stably expressing surface bound OKT3 were transfected with gagpol, RD-PRO env, the transfer vector or all three plasmids along with rev. The subsequent supernatant was applied to primary human T-cells. The T-cells were studied by flow-cytometry with the following parameters: CD25 to measure T-cell activation; anti-Fc to detect transgene which was a CAR with an Fc spacer; ki67 to determine cells in cycle. Only conditions where gagpol was supplied resulted in significant mitogenic stimulation. Only the condition where all plasmids were supplied (along with rev) resulted in mitogenic stimulation of T-cells and transduction.

FIG. 4—Demonstration that different lentiviral pseudotyping supports the mitogenic effect. 293T cells stably expressing the membrane bound OKT3 were transfected with a lentiviral transfer vector, lentiviral gagpol, rev and different env plasmids: namely VSV-G, RD-PRO, Ampho, GALV and Measles M/H. The subsequent supernatant was applied to primary human T-cells. The cells were subsequently stained with ki67 and studied by flow-cytometry. All pseudotypes supported the mitogenic effect, although the effect seemed reduced with Measles pseudotyping.

FIG. 5—Demonstration that mitogenic stimulation and transduction of T cells is achieved with a gamma-retroviral vector. 293T cells stably expressing membrane bound OKT3 were transfected with a gamma-retroviral transfer vector coding for a CAR, gamma-retroviral gagpol expression plasmid and an RD114 expression plasmid. Subsequent supernant was applied to primary human T-cells. The T-cells were subsequently stained with anti-Fc, anti-CD25 and ki67 and studied by flow-cytometry. Although no mitogenic stimulus was applied, T-cells were activated, cycling and were expressing transgene.

FIG. 6—Demonstration that two different mitogenic stimuli can be incorporated into the viral vector and that an anti-CD3/TCR stimulus along with an anti-CD28 stimulus has an improved effect compared to anti-CD3/TCR alone.

FIG. 7—Low resolution microscopy of T-cells stimulated with different lentiviral vectors generated from 293T cells expressing different elements on their cell surface.

FIG. 8—Activation of CD4 and CD8 T cells following transduction with lentiSTIM vectors displaying different combinations of mitogenic and cytokine peptides. Activation is determined by CD25 expression at 120-hours post-transduction.

FIG. 9—Proliferation of CD4 and CD8 T cells following transduction with lentiSTIM vectors displaying different combinations of mitogenic and cytokine peptides. Proliferation is determined by Ki67 expression at 120-hours post-transduction.

FIG. 10—Expansion of T cells following transduction with lentiSTIM vectors displaying different combinations of mitogenic and cytokine peptides. Expansion is determined by absolute cell counts at 120-hours post-transduction.

FIG. 11—Examining the T cell subset phenotype of PBMCs activated with either lentiSTIM vectors expressing anti-CD3 and anti-CD28 antibodies, or beads coated with anti-CD3 and anti-CD28 antibodies. NM-LV=non-modified lentivirus; STIM-LV=lentiSTIM vector; Tem=effector memory T cells; Tom=central memory T cells; Tscm=stem memory T cells; and Tn=naïve T cells.

### SUMMARY OF ASPECTS OF THE INVENTION

The present invention is based on the finding that it is possible to incorporate a mitogenic stimulus, and/or a cytokine stimulus, into a retroviral or lentiviral capsid, such that the virus both activates and transduces T cells. This removes the need to add vector, mitogen and cytokines. The invention involves including a mitogenic transmembrane protein and/or a cytokine-based transmembrane protein in the producer or packaging cell, which get(s) incorporated into the retrovirus when it buds from the producer/packaging cell membrane. The mitogenic transmembrane protein and/or a cytokine-based transmembrane protein is/are expressed as a separate cell surface molecule on the producer cell rather than being part of the viral envelope glycoprotein. This means that the reading frame of the viral envelope is unaffected, which therefore preserves functional integrity and viral titre.

Thus in a first aspect the present invention provides a retroviral or lentiviral vector having a viral envelope which comprises:
(i) a mitogenic T-cell activating transmembrane protein which comprises a mitogenic domain and a transmembrane domain; and/or
(ii) a cytokine-based T-cell activating transmembrane protein which comprises a cytokine domain and a transmembrane domain wherein the mitogenic or cytokine-based T-cell activating transmembrane protein is not part of a viral envelope glycoprotein

The retroviral or lentiviral vector may comprise a separate viral envelope glycoprotein, encoded by an env gene.

Thus there is provided a retroviral or lentiviral vector having a viral envelope which comprises:
(i) a viral envelope glycoprotein; and
(ii) a mitogenic T-cell activating transmembrane protein having the structure:

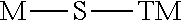

in which M is a mitogenic domain; S is an optional spacer and TM is a transmembrane domain; and/or
(iii) a cytokine-based T-cell activating transmembrane protein which comprises a cytokine domain and a transmembrane domain.

The mitogenic T-cell activating transmembrane protein and/or cytokine-based T-cell activating transmembrane protein are not part of the viral envelope glycoprotein. They exist as separate proteins in the viral envelope and are encoded by separate genes.

The mitogenic T-cell activating transmembrane protein may have the structure:

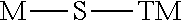

in which M is a mitogenic domain; S is an optional spacer and TM is a transmembrane domain.

The mitogenic T-cell activating transmembrane protein may bind an activating T-cell surface antigen such as CD3, CD28, CD134 or CD137. The mitogenic T-cell activating transmembrane protein may comprise an agonist for such an activating T-cell surface antigen.

The mitogenic T-cell activating transmembrane protein may comprise the binding domain from an antibody such as OKT3, 15E8, TGN1412; or a costimulatory molecule such as OX40L or 41BBL.

The viral vector may comprise two or more mitogenic T-cell activating transmembrane proteins in the viral envelope. For example, the viral vector may comprise a first mitogenic T-cell activating transmembrane protein which binds CD3 and a second mitogenic T-cell activating transmembrane protein which binds CD28.

The cytokine-based T-cell activating transmembrane protein may, for example, comprise a cytokine selected from IL2, IL7 and IL15.

In particular there is provided a retroviral or lentiviral vector having a viral envelope which comprises:
(ia) a first mitogenicT-cell activating transmembrane protein which binds CD3; and
(ib) a second mitogenic T-cell activating transmembrane protein which binds CD28.

There is also provided a retroviral or lentiviral vector having a viral envelope which comprises:
(ia) a first mitogenicT-cell activating transmembrane protein which binds CD3;
(ib) a second mitogenic T-cell activating transmembrane protein which binds CD28; and
(ii) a cytokine-based T-cell activating transmembrane protein which comprises IL2.

There is also provided a retroviral or lentiviral vector having a viral envelope which comprises:
(ia) a first mitogenicT-cell activating transmembrane protein which binds CD3;
(ib) a second mitogenic T-cell activating transmembrane protein which binds CD28;
(iia) a cytokine-based T-cell activating transmembrane protein which comprises IL7; and
(iib) a cytokine-based T-cell activating transmembrane protein which comprises IL15.

The viral vector may comprise a heterologous viral envelope glycoprotein giving a pseudotyped viral vector. For example, the viral envelope glycoprotein may be derived from RD114 or one of its variants, VSV-G, Gibbon-ape leukaemia virus (GALV), or is the Amphotropic envelope, Measles envelope or baboon retroviral envelope glycoprotein.

In a second embodiment of the first aspect of the invention, the viral envelope of the viral vector may also comprise:
(iv) a tagging protein which comprises:
   a binding domain which binds to a capture moiety
   a spacer; and
   a transmembrane domain
which tagging protein facilitates purification of the viral vector from cellular supernatant via binding of the tagging protein to the capture moiety.

The binding domain of the tagging protein may comprise one or more streptavidin-binding epitope(s). The streptavidin-binding epitope(s) may be a biotin mimic, such as a biotin mimic which binds streptavidin with a lower affinity than biotin, so that biotin may be used to elute streptavidin-captured retroviral vectors produced by the packaging cell.

Examples of suitable biotin mimics include: StreptagII (SEQ ID NO: 36), Flankedcostretag (SEQ ID NO: 37) and ccstreptag (SEQ ID NO:38).

The viral vector of the first aspect of the invention may comprise a nucleic acid sequence encoding a T-cell receptor or a chimeric antigen receptor.

The viral vector may be a virus-like particle (VLP).

In a second aspect, the present invention provides a host cell which expresses, at the cell surface,
(ii) a mitogenic T-cell activating transmembrane protein comprising a mitogenic domain and a transmembrane domain; and/or
(ii) a cytokine-based T-cell activating transmembrane protein which comprises a cytokine domain and a transmembrane domain
such that a retroviral or lentiviral vector produced by the packaging cell is as defined in the first aspect of the invention.

In a second embodiment of the second aspect of the invention, the host cell may also express, at the cell surface:
(iii) a tagging protein which comprises:
   a binding domain which binds to a capture moiety;
and
   a transmembrane domain
which tagging protein facilitates purification of the viral vector from cellular supernatant via binding of the tagging protein to the capture moiety,
such that a retroviral or lentiviral vector produced by the packaging cell is as defined in the second embodiment of the first aspect of the invention.

The tagging protein may also comprise a spacer between the binding domain and the transmembrane domain.

The term host cell may be a packaging cell or a producer cell.

A packaging cell may comprise one or more of the following genes: gag, pol, env and/or rev.

A producer cell comprises gag, pol, env and optionally rev genes and also comprises a retroviral or lentiviral genome.

In this respect, the host cell may be any suitable cell line stably expressing mitogenic and/or cytokine transmembrane proteins. It may be transiently transfected with transfer vector, gagpol, env (and rev in the case of a lentivirus) to produce replication incompetent retroviral/lentiviral vector.

In a third aspect there is provided a method for making a host cell according to the second aspect of the invention, which comprises the step of transducing or transfecting a cell with a nucleic acid encoding a mitogenic T-cell activating transmembrane protein and/or a cytokine-based T-cell activating transmembrane protein.

In a fourth aspect there is provided a method for producing a viral vector according to the first aspect of the invention which comprises the step of expressing a retroviral or lentiviral genome in a cell according to the second aspect of the invention.

In a fifth aspect, there is provided a method for making an activated transgenic T-cell or natural killer (NK) cell, which comprises the step of transducing a T or NK cell with a viral vector according to the first aspect of the invention, such that the T-cell or NK cell is activated by the one or more mitogenic T-cell activating transmembrane protein(s) and/or the one or more cytokine-based T-cell activating transmembrane protein(s).

In a sixth aspect, there is provided a kit for making a retroviral or lentiviral vector as defined in the first aspect of the invention, which comprises:
(i) a host cell as defined in the second aspect of the invention;
(ii) nucleic acids comprising gag,pol,env and optionally rev; and
(iii) a retroviral genome.

There is also provided is provided a kit for making a retroviral or lentiviral vector as defined in the first aspect of the invention, which comprises:
(i) a packaging cell as defined in the second aspect of the invention; and
(ii) a retroviral genome.

There is also provided a kit for making a packaging cell according to the second embodiment of the second aspect of the invention which comprises:
(i) one or more nucleic acid(s) encoding a mitogenic T-cell activating transmembrane protein and/or a cytokine-based T-cell activating transmembrane protein; and
(ii) nucleic acids comprising retroviral gag, pol and env genes.

There is also provided a kit for making a producer cell according to the second aspect of the invention, which comprises:
(i) one or more nucleic acid(s) encoding a mitogenic T-cell activating transmembrane protein and/or a cytokine-based T-cell activating transmembrane protein;
(ii) nucleic acids comprising retroviral gag, pol and env genes; and
(iii) a retroviral or lentiviral vector genome

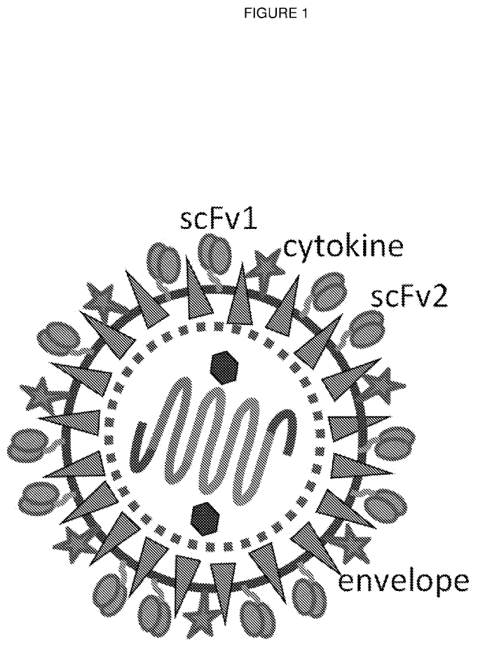

The invention therefore provides a viral vector with a built-in mitogenic stimulus and/or cytokine stimulus (see FIG. 1). The vector has the capability to both stimulate the T-cell and to also effect gene insertion. This has a number of advantages: (1) it simplifies the process of T-cell engineering, as only one component needs to be added; (2) it avoids removal of beads and the associated reduction in yield as the virus is labile and does not have to be removed. (3) it reduces the cost of T-cell engineering as only one component needs to be manufactured; (4) it allows greater design flexibility: each T-cell engineering process will involve making a gene-transfer vector, the same product can also be made with a mitogenic stimulus to “fit” the product; (5) it allows for a shortened production process: in soluble antigen/bead-based approaches the mitogen and the vector are typically given sequentially separated by one, two or sometimes three days, this can be avoided with the retroviral vector of the present invention since mitogenic stimulation and viral entry are synchronized and simultaneous; (6) it is easier to engineer as there is no need to test a lot of different fusion proteins for expression and functionality; (7) it is possible to add more than one signal at the same time; and (8) it is possible to regulate the expression and/or expression levels of each signal/protein separately.

Since the mitogenic stimulus and/or cytokine stimulus are provided on a molecule which is separate from the viral envelope glycoprotein, integrity of the viral envelope glycoprotein is maintained and there is no negative impact on viral titre.

### DETAILED DESCRIPTION

### Retroviruses

Retroviruses are double stranded RNA enveloped viruses mainly characterized by the ability to “reverse-transcribe” their genome from RNA to DNA. Virions measure 100-120 nm in diameter and contain a dimeric genome of identical positive RNA strands complexed with the nucleocapsid proteins. The genome is enclosed in a proteic capsid that also contains enzymatic proteins, namely the reverse transcriptase, the integrase and proteases, required for viral infection. The matrix proteins form a layer outside the capsid core that interacts with the envelope, a lipid bilayer derived from the host cellular membrane, which surrounds the viral core particle. Anchored on this bilayer, are the viral envelope glycoproteins responsible for recognizing specific receptors on the host cell and initiating the infection process. Envelope proteins are formed by two subunits, the transmembrane (TM) that anchors the protein into the lipid membrane and the surface (SU) which binds to the cellular receptors.

Based on the genome structure, retroviruses are classified into simple retroviruses, such as MLV and murine leukemia virus; or complex retroviruses, such as HIV and EIAV. Retroviruses encode four genes: gag (group specific antigen), pro (protease), pol (polymerase) and env (envelope). The gag sequence encodes the three main structural proteins: the matrix protein, nucleocapsid proteins, and capsid protein. The pro sequence encodes proteases responsible for cleaving Gag and Gag-Pol during particle assembly, budding and maturation. The pol sequence encodes the enzymes reverse transcriptase and integrase, the former catalyzing the reverse transcription of the viral genome from RNA to DNA during the infection process and the latter responsible for integrating the proviral DNA into the host cell genome. The env sequence encodes for both SU and TM subunits of the envelope glycoprotein. Additionally, retroviral genome presents non-coding cis-acting sequences such as: two LTRs (long terminal repeats), which contain elements required to drive gene expression, reverse transcription and integration into the host cell chromosome; a sequence named packaging signal (w) required for specific packaging of the viral RNA into newly forming virions; and a polypurine tract (PPT) that functions as the site for initiating the positive strand DNA synthesis during reverse transcription. In addition to gag, pro, pol and env, complex retroviruses, such as lentiviruses, have accessory genes including vif, vpr, vpu, nef, tat and rev that regulate viral gene expression, assembly of infectious particles and modulate viral replication in infected cells.

During the process of infection, a retrovirus initially attaches to a specific cell surface receptor. On entry into the susceptible host cell, the retroviral RNA genome is then copied to DNA by the virally encoded reverse transcriptase which is carried inside the parent virus. This DNA is transported to the host cell nucleus where it subsequently integrates into the host genome. At this stage, it is typically referred to as the provirus.

The provirus is stable in the host chromosome during cell division and is transcribed like other cellular proteins. The provirus encodes the proteins and packaging machinery required to make more virus, which can leave the cell by a process known as “budding”.

When enveloped viruses, such as retrovirus and lentivirus, bud out of the host cells, they take part of the host cell lipidic membrane. In this way, host-cell derived membrane proteins become part of the retroviral particle. The present invention utilises this process in order to introduce proteins of interest into the envelope of the viral particle.

### Retroviral Vectors

Retroviruses and lentiviruses may be used as a vector or delivery system for the transfer of a nucleotide of interest (NOI), or a plurality of NOIs, to a target cell. The transfer can occur in vitro, ex vivo or in vivo. When used in this fashion, the viruses are typically called viral vectors.

In the viral vectors of the present invention, the NOI may encode a T cell receptor or a chimeric antigen receptor and/or a suicide gene.

Gamma-retroviral vectors, commonly designated retroviral vectors, were the first viral vector employed in gene therapy clinical trials in 1990 and are still one of the most used. More recently, the interest in lentiviral vectors, derived from complex retroviruses such as the human immunodeficiency virus (HIV), has grown due to their ability to transduce non-dividing cells. The most attractive features of retroviral and lentiviral vectors as gene transfer tools include the capacity for large genetic payload (up to 9 kb), minimal patient immune response, high transducing efficiency in vivo and in vitro, and the ability to permanently modify the genetic content of the target cell, sustaining a long-term expression of the delivered gene.

The retroviral vector can be based on any suitable retrovirus which is able to deliver genetic information to eukaryotic cells. For example, the retroviral vector may be an alpharetroviral vector, a gammaretroviral vector, a lentiviral vector or a spumaretroviral vector. Such vectors have been used extensively in gene therapy treatments and other gene delivery applications.

The viral vector of the present invention may be a retroviral vector, such as a gamma-retroviral vector. The viral vector may be based on human immunodeficiency virus.

The viral vector of the present invention may be a lentiviral vector. The vector may be based on a non-primate lentivirus such as equine infectious anemia virus (EIAV).

The viral vector of the invention comprises a mitogenic T-cell activating transmembrane protein and/or a cytokine-based T-cell activating transmembrane protein in the viral envelope, as illustrated in FIG. 1.

The mitogenic T-cell activating transmembrane protein and/or cytokine-based T-cell activating transmembrane protein is/are derived from the host cell membrane, as explained above.

### Virus-Like Particles (VLPs)

For retroviral and lentiviral vectors, the expression of the Gag precursor is sufficient to mediate virion assembly and release. Gag proteins, and even fragments of Gag, have been shown competent to assemble in vitro to form various structures that resemble virion cores. These particles that are devoid of viral genetic material, and are hence non-infectious, are called virus-like particles (VLPs). Like with complete viral particles they contain an outer viral envelope made of the host cell lipid-bi-layer (membrane), and hence contain host cell transmembrane proteins.

The viral vector of the first aspect of the invention may be or comprise a virus-like particle.

### Nucleotide of Interest (NOI)

The viral vector of the present invention is capable of delivering a nucleotide of interest (NOI) to a target cell, such as a T cell or a natural killer (NK) cell.

The NOI may encode all or part of a T-cell receptor (TCR) or a chimeric antigen receptor (CAR) and/or a suicide gene.

CARs, are chimeric type I trans-membrane proteins which connect an extracellular antigen-recognizing domain (binder) to an intracellular signalling domain (endodomain). The binder is typically a single-chain variable fragment (scFv) derived from a monoclonal antibody (mAb), but it can be based on other formats which comprise an antibody-like antigen binding site. A spacer domain is usually necessary to isolate the binder from the membrane and to allow it a suitable orientation. A trans-membrane domain anchors the protein in the cell membrane. A CAR may comprise or associate with an intracellular T-cell signalling domain or endodomain.

CAR-encoding nucleic acids may be transferred to cells, such a T cells, using the retroviral or lentiviral vector of the present invention. In this way, a large number of cancer-specific T cells can be generated for adoptive cell transfer. When the CAR binds the target-antigen, this results in the transmission of an activating signal to the T-cell it is expressed on. Thus the CAR directs the specificity and cytotoxicity of the T cell towards tumour cells expressing the targeted antigen.

A suicide gene encodes a polypeptide which enable the cells expressing such a polypeptide to be deleted, for example by triggering apoptosis. An example of a suicide gene is described in WO2013/153391.

### Host Cell

In a second aspect, the invention provides a host cell which expresses a mitogenic T-cell activating transmembrane protein or a cytokine-based T-cell activating transmembrane protein at the cell surface.

The host cell may be for the production of viral vectors according to the first aspect of the invention.

The host cell may be a packaging cell and comprise one or more of the following genes: gag, pol, env and rev.

A packaging cell for a retroviral vector may comprise gag, pol and env genes.

A packaging cell for a lentiviral vector may comprises gag, pol, env and rev genes.

The host cell may be a producer cell and comprise gag, pol, env and optionally rev genes and a retroviral or lentiviral vector genome.

In a typical recombinant retroviral or lentiviral vector for use in gene therapy, at least part of one or more of the gag-pol and env protein coding regions may be removed from the virus and provided by the packaging cell. This makes the viral vector replication-defective as the virus is capable of integrating its genome into a host genome but the modified viral genome is unable to propagate itself due to a lack of structural proteins.

Packaging cells are used to propagate and isolate quantities of viral vectors i.e to prepare suitable titres of the retroviral vector for transduction of a target cell.

In some instances, propagation and isolation may entail isolation of the retroviral gagpol and env (and in the case of lentivirus, rev) genes and their separate introduction into a host cell to produce a packaging cell line. The packaging cell line produces the proteins required for packaging retroviral DNA but it cannot bring about encapsidation due to the lack of a psi region. However, when a recombinant vector carrying a psi region is introduced into the packaging cell line, the helper proteins can package the psi-positive recombinant vector to produce the recombinant virus stock.

A summary of the available packaging lines is presented in “Retroviruses” (1997 Cold Spring Harbour Laboratory Press Eds: J M Coffin, SM Hughes, HE Varmus pp 449).

Packaging cells have also been developed in which the gag, pol and env (and, in the case of lentiviral vectors, rev) viral coding regions are carried on separate expression plasmids that are independently transfected into a packaging cell line, so that three recombinant events are required for wild type viral production.

Transient transfection avoids the longer time required to generate stable vector-producing cell lines and is used if the vector or retroviral packaging components are toxic to cells. Components typically used to generate retroviral/lentivial vectors include a plasmid encoding the Gag/Pol proteins, a plasmid encoding the Env protein (and, in the case of lentiviral vectors, the rev protein), and the retroviral/lentiviral vector genome. Vector production involves transient transfection of one or more of these components into cells containing the other required components.

The packaging cells of the present invention may be any mammalian cell type capable of producing retroviral/lentiviral vector particles. The packaging cells may be 293T-cells, or variants of 293T-cells which have been adapted to grow in suspension and grow without serum.

The packaging cells may be made by transient transfection with
a) the transfer vector
b) a gagpol expression vector
c) an env expression vector. The env gene may be a heterologous, resulting in a pseudotyped retroviral vector. For example, the env gene may be from RD114 or one of its variants, VSV-G, the Gibbon-ape leukaemia virus (GALV), the Amphotropic envelope or Measles envelope or baboon retroviral envelope glycoprotein.

In the case of lentiviral vector, transient transfection with a rev vector is also performed.

### Mitogenic T-Cell Activating Transmembrane Protein

The viral vector of the present invention may comprise a mitogenic T-cell activating transmembrane protein in the viral envelope. The mitogenic T-cell activating transmembrane protein is derived from the host cell during retroviral vector production. The mitogenic T-cell activating transmembrane protein is made by the packaging cell and expressed at the cell surface. When the nascent retroviral vector buds from the host cell membrane, the mitogenic T-cell activating transmembrane protein is incorporated in the viral envelope as part of the packaging cell-derived lipid bilayer.

The term “host-cell derived” indicates that the mitogenic T-cell activating transmembrane protein is derived from the host cell as described above and is not produced as a fusion or chimera from one of the viral genes, such as gag, which encodes the main structural proteins; or env, which encodes the envelope protein.

Envelope proteins are formed by two subunits, the transmembrane (TM) that anchors the protein into the lipid membrane and the surface (SU) which binds to the cellular receptors. The packaging-cell derived mitogenic T-cell activating transmembrane protein of the present invention does not comprise the surface envelope subunit (SU).

The mitogenic T-cell activating transmembrane protein may comprise one of the following sequences, or a variant thereof.

(OKT3-CD8STK-TM-A)
SEQ ID No. 1
METDTLLLWVLLLWVPGSTGQVQLQQSGAELARPGASVKMSCKASGYTFT
RYTMHWVKQRPGQGLEWIGYINPSRGYTNYNQKFKDKATLTTDKSSSTAY
MQLSSLTSEDSAVYYCARYYDDHYCLDYWGQGTTLTVSSSGGGGSGGGGS
GGGGSQIVLTQSPAIMSASPGEKVTMTCSASSSVSYMNWYQQKSGTSPKR
WIYDTSKLASGVPAHFRGSGSGTSYSLTISGMEAEDAATYYCQQWSSNPF
TFGSGTKLEINRSDPTTTPAPRPPTPAPTIASQPLSLRPEACRPAAGGAV
HTRGLDFACDIYIWAPLAGTCGVLLLSLVITLYCNHRNRRRVCKCPRPVV
(15E8-CD8STK-TM-A)
SEQ ID No. 2
METDTLILWVLLLLVPGSTGQVQLKESGPGLVAPSQSLSITCTVSGFSLT
SYGVHWVRQPPGKGLEWLGVIWAGGSTNYNSALMSRLSISKDNSKSQVFL
KMNSLQTDDTAMYYCARDKRAPGKLYYGYPDYWGQGTTLTVSSGGGGSGG
GGSGGGGSDIVLTQSPASLAVSLGQRATISCRASESVEYYVTSLMQVVYQ
QKPGQPPKLLIYAASNVESGVPARFSGSGSGTDFSLNIHPVEEDDIAMYF
CQQTRKVPSTFGGGTKLEIKRSDPTTTPAPRPPTPAPTIASQPLSLRPEA
CRPAAGGAVHTRGLDFACDIYIWAPLAGTCGVLLLSLVITLYCNHRNRRR
VCKCPRPVV
(TGN1412-CD8STK-TM-A)
SEQ ID No. 3
METDTLILWVLLLLVPGSTGQVQLVQSGAEVKKPGASVKVSCKASGYTFT
SYYIHWVRQAPGQGLEWIGCIYPGNVNTNYNEKFKDRATLTVDTSISTAY
MELSRLRSDDTAVYFCTRSHYGLDWNFDVWGQGTTVTVSSGGGGSGGGGS
GGGGSDIQMTQSPSSLSASVGDRVTITCHASQNIYVWLNWYQQKPGKAPK
WYKASNLHTGVPSRFSGSGSGTDFTLTISSLQPEDFATYYCQQGQTYPYT
FGGGTKVEIKRSDPTTTPAPRPPTPAPTIASQPLSLRPEACRPAAGGAVH
TRGLDFACDIYIWAPLAGTCGVLLLSLVITLYCNHRNRRRVCKCPRPVV

The mitogenic T-cell activating transmembrane protein may comprise a variant of the sequence shown as SEQ ID No. 1, 2 or 3 having at least 80, 85, 90, 95, 98 or 99% sequence identity, provided that the variant sequence is a mitogenic T-cell activating transmembrane protein having the required properties i.e. the capacity to activate a T cell when present in the envelope protein of a retroviral vector.

Methods of sequence alignment are well known in the art and are accomplished using suitable alignment programs. The % sequence identity refers to the percentage of amino acid or nucleotide residues that are identical in the two sequences when they are optimally aligned. Nucleotide and protein sequence homology or identity may be determined using standard algorithms such as a BLAST program (Basic Local Alignment Search Tool at the National Center for Biotechnology Information) using default parameters, which is publicly available at http://blast.ncbi.nlm.nih.gov. Other algorithms for determining sequence identity or homology include: LALIGN (http://www.ebi.ac.uk/Tools/psa/lalign/and http://www.ebi.ac.uk/Tools/psa/lalign/nucleotide.html), AMAS (Analysis of Multiply Aligned Sequences, at http://www.compbio.dundee.ac.uk/Software/Amas/amas.html), FASTA (http://www.ebi.ac.uk/Tools/sss/fasta/) Clustal Omega (http://www.ebi.ac.uk/Tools/msa/clustalo/), SIM (http://web.expasy.org/sim/), and EMBOSS Needle (http://www.ebi.ac.uk/Tools/psa/emboss_needle/nucleotide.html).

The mitogenic T-cell activating transmembrane protein may have the structure:

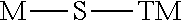

in which M is a mitogenic domain; S is an optional spacer domain and TM is a transmembrane domain.

### Mitogenic Domain

The mitogenic domain is the part of the mitogenic T-cell activating transmembrane protein which causes T-cell activation. It may bind or otherwise interact, directly or indirectly, with a T cell, leading to T cell activation. In particular, the mitogenic domain may bind a T cell surface antigen, such as CD3, CD28, CD134 and CD137.

CD3 is a T-cell co-receptor. It is a protein complex composed of four distinct chains. In mammals, the complex contains a CD3γ chain, a CD30 chain, and two CD3& chains. These chains associate with the T-cell receptor (TCR) and the Z-chain to generate an activation signal in T lymphocytes. The TCR, Z-chain, and CD3 molecules together comprise the TCR complex.

The mitogenic domain may bind to CD3 & chain.

CD28 is one of the proteins expressed on T cells that provide co-stimulatory signals required for T cell activation and survival. T cell stimulation through CD28 in addition to the T-cell receptor (TCR) can provide a potent signal for the production of various interleukins (IL-6 in particular).

CD134, also known as OX40, is a member of the TNFR-superfamily of receptors which is not constitutively expressed on resting naïve T cells, unlike CD28. OX40 is a secondary costimulatory molecule, expressed after 24 to 72 hours following activation; its ligand, OX40L, is also not expressed on resting antigen presenting cells, but is following their activation. Expression of OX40 is dependent on full activation of the T cell; without CD28, expression of OX40 is delayed and of fourfold lower levels.

CD137, also known as 4-1BB, is a member of the tumor necrosis factor (TNF) receptor family. CD137 can be expressed by activated T cells, but to a larger extent on CD8 than on CD4 T cells. In addition, CD137 expression is found on dendritic cells, follicular dendritic cells, natural killer cells, granulocytes and cells of blood vessel walls at sites of inflammation. The best characterized activity of CD137 is its costimulatory activity for activated T cells. Crosslinking of CD137 enhances T cell proliferation, IL-2 secretion survival and cytolytic activity.

The mitogenic domain may comprise all or part of an antibody or other molecule which specifically binds a T-cell surface antigen. The antibody may activate the TCR or CD28. The antibody may bind the TCR, CD3 or CD28. Examples of such antibodies include: OKT3, 15E8 and TGN1412. Other suitable antibodies include:
Anti-CD28: CD28.2, 10F3
Anti-CD3/TCR: UCHT1, YTH12.5, TR66

The mitogenic domain may comprise the binding domain from OKT3, 15E8, TGN1412, CD28.2, 10F3, UCHT1, YTH12.5 or TR66.

The mitogenic domain may comprise all or part of a co-stimulatory molecule such as OX40L and 41BBL. For example, the mitogenic domain may comprise the binding domain from OX40L or 41BBL.

OKT3, also known as Muromonab-CD3 is a monoclonal antibody targeted at the CD38 chain. It is clinically used to reduce acute rejection in patients with organ transplants. It was the first monoclonal antibody to be approved for clinical use in humans. The CDRs of OKT3 are as follows:

CDRH1:
(SEQ ID No. 4)
GYTFTRY
CDRH2:
(SEQ ID No. 5)
NPSRGY
CDRH3:
(SEQ ID No. 6)
YYDDHYCLDY
CDRL1:
(SEQ ID No. 7)
SASSSVSYMN
CDRL2:
(SEQ ID No. 8)
DTSKLAS
CDRL3:
(SEQ ID No. 9)
QQWSSNPFT

15E8 is a mouse monoclonal antibody to human CD28. Its CDRs are as follows:

CDRH1:
(SEQ ID No. 10)
GFSLTSY
CDRH2:
(SEQ ID No. 11)
WAGGS
CDRH3:
(SEQ ID No. 12)
DKRAPGKLYYGYPDY
CDRL1:
(SEQ ID No. 13)
RASESVEYYVTSLMQ
CDRL2:
(SEQ ID No. 14)
AASNVES
CDRL3:
(SEQ ID No. 15)
QQTRKVPST

TGN1412 (also known as CD28-SuperMAB) is a humanised monoclonal antibody that not only binds to, but is a strong agonist for, the CD28 receptor. Its CDRs are as follows.

CDRH1:
(SEQ ID No. 16)
GYTFSY
CDRH2:
(SEQ ID No. 17)
YPGNVN
CDRH3:
(SEQ ID No. 18)
SHYGLDWNFDV
CDRL1:
(SEQ ID No. 19)
HASQNIYVLN
CDRL2:
(SEQ ID No. 20)
KASNLHT
CDRL3:
(SEQ ID No. 21)
QQGQTYPYT

OX40L is the ligand for CD134 and is expressed on such cells as DC2s (a subtype of dendritic cells) enabling amplification of Th2 cell differentiation. OX40L has also been designated CD252 (cluster of differentiation 252).

OX40L sequence
(SEQ ID No. 22)
MERVQPLEENVGNAARPRFERNKLLLVASVIQGLGLLLCFTYICLHFSAL
QVSHRYPRIQSIKVQFTEYKKEKGFILTSQKEDEIMKVQNYLISLKGYFS
QEVNISLHYQKDEEPLFQLKKVRSVNSLMVASLTYKDKVYLNVTTDNTSL
DDFHVNGGELILIHQNPGEFCVL

41BBL is a cytokine that belongs to the tumour necrosis factor (TNF) ligand family. This transmembrane cytokine is a bidirectional signal transducer that acts as a ligand for 4-1BB, which is a costimulatory receptor molecule in T lymphocytes. 41BBL has been shown to reactivate anergic T lymphocytes in addition to promoting T lymphocyte proliferation.

41BBL sequence
(SEQ ID No. 23)
MEYASDASLDPEAPWPPAPRARACRVLPWALVAGLLLLLLLAAACAVFLA
CPWAVSGARASPGSAASPRLREGPELSPDDPAGLLDLRQGMFAQLVAQNV
LLIDGPLSWYSDPGLAGVSLTGGLSYKEDTKELVVAKAGVYYVFFQLELR
RVVAGEGSGSVSLALHLQPLRSAAGAAALALTVDLPPASSEARNSAFGFQ
GRLLHLSAGQRLGVHLHTEARARHAWQLTQGATVLGLFRVTPEIPAGLPS
PRSE

### Spacer Domain

The mitogenic T-cell activating transmembrane protein and/or cytokine-based T-cell activating transmembrane protein may comprise a spacer sequence to connect the antigen-binding domain with the transmembrane domain. A flexible spacer allows the antigen-binding domain to orient in different directions to facilitate binding.

The spacer sequence may, for example, comprise an IgG1 Fc region, an IgG1 hinge or a human CD8 stalk or the mouse CD8 stalk. The spacer may alternatively comprise an alternative linker sequence which has similar length and/or domain spacing properties as an IgG1 Fc region, an IgG1 hinge or a CD8 stalk. A human IgG1 spacer may be altered to remove Fc binding motifs.

Examples of amino acid sequences for these spacers are given below:

SEQ ID No. 24 (hinge-CH2CH3 of human IgG1)
AEPKSPDKTHTCPPCPAPPVAGPSVFLFPPKPKDTLMIARTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKS
RWQQGNVFSCSVMHEALHNHYTQKSLSLSPGKKD
SEQ ID No. 25 (human CD8 stalk):
TTTPAPRPPTPAPTIASQPLSLRPEACRPAAGGAVHTRGLDFACDI
SEQ ID No. 26 (human IgG1 hinge):
AEPKSPDKTHTCPPCPKDPK
SEQ ID No. 27 (CD2 ectodomain)
KEITNALETWGALGQDINLDIPSFQMSDDIDDIKWEKTSDKKKIAQFRKE
KETFKEKDTYKLFKNGTLKIKHLKTDDQDIYKVSIYDTKGKNVLEKIFDL
KIQERVSKPKISWTCINTTLTCEVMNGTDPELNLYQDGKHLKLSQRVITH
KWTTSLSAKFKCTAGNKVSKESSVEPVSCPEKGLD
SEQ ID no. 28 (CD34 ectodomain)
SLDNNGTATPELPTQGTFSNVSTNVSYQETTTPSTLGSTSLHPVSQHGNE
ATTNITETTVKFTSTSVITSVYGNTNSSVQSQTSVISTVFTTPANVSTPE
TTLKPSLSPGNVSDLSTTSTSLATSPTKPYTSSSPILSDIKAEIKCSGIR
EVKLTQGICLEQNKTSSCAEFKKDRGEGLARVLCGEEQADADAGAQVCSL
LLAQSEVRPQCLLLVLANRTEISSKLQLMKKHQSDLKKLGILDFTEQDVA
SHQSYSQKT

The spacer sequence may be derived from a human protein. The spacer sequence may not be derived from a viral protein. In particular, the spacer sequence may not be, be derived from, or comprise part of the surface envelope subunit (SU) of a retroviral env protein.

### Transmembrane Domain

The transmembrane domain is the sequence of the mitogenic T-cell activating transmembrane protein and/or cytokine-based T-cell activating transmembrane protein that spans the membrane. The transmembrane domain may comprise a hydrophobic alpha helix. The transmembrane domain may be derived from CD28, which gives good receptor stability.

The transmembrane domain may be derived from a human protein. The transmembrane domain may not be derived from a viral protein. In particular, the transmembrane domain may not be, be derived from, or comprise part of the transmembrane envelope subunit (TM) of a retroviral env protein.

An alternative option to a transmembrane domain is a membrane-targeting domain such as a GPI anchor.

GPI anchoring is a post-translational modification which occurs in the endoplasmic reticulum. Preassembled GPI anchor precursors are transferred to proteins bearing a C-terminal GPI signal sequence. During processing, the GPI anchor replaces the GPI signal sequence and is linked to the target protein via an amide bond. The GPI anchor targets the mature protein to the membrane.

The present tagging protein may comprise a GPI signal sequence.

### Cytokine-Based T-Cell Activating Transmembrane Protein

The viral vector of the present invention may comprise a cytokine-based T-cell activating transmembrane protein in the viral envelope. The cytokine-based T-cell activating transmembrane protein is derived from the host cell during viral vector production. The cytokine-based T-cell activating transmembrane protein is made by the host cell and expressed at the cell surface. When the nascent viral vector buds from the host cell membrane, the cytokine-based T-cell activating transmembrane protein is incorporated in the viral envelope as part of the packaging cell-derived lipid bilayer.

The cytokine-based T-cell activating transmembrane protein is not produced from one of the viral genes, such as gag, which encodes the main structural proteins, or env, which encodes the envelope protein.

The cytokine-based T-cell activating transmembrane protein may comprise a cytokine domain and a transmembrane domain. It may have the structure C-S-TM, where C is the cytokine domain, S is an optional spacer domain and TM is the transmembrane domain. The spacer domain and transmembrane domains are as defined above.

### Cytokine Domain

The cytokine domain may comprise part or all of a T-cell activating cytokine, such as from IL2, IL7 and IL15. The cytokine domain may comprise part of the cytokine, as long as it retains the capacity to bind its particular receptor and activate T-cells.

IL2 is one of the factors secreted by T cells to regulate the growth and differentiation of T cells and certain B cells. IL2 is a lymphokine that induces the proliferation of responsive T cells. It is secreted as a single glycosylated polypeptide, and cleavage of a signal sequence is required for its activity. Solution NMR suggests that the structure of IL2 comprises a bundle of 4 helices (termed A-D), flanked by 2 shorter helices and several poorly defined loops. Residues in helix A, and in the loop region between helices A and B, are important for receptor binding. The sequence of IL2 is shown as SEQ ID No. 29.

SEQ ID No. 29
MYRMQLLSCIALSLALVTNSAPTSSSTKKTQLQLEHLLLDLQMILNGINN
YKNPKLTRMLTFKFYMPKKATELKHLQCLEEELKPLEEVLNLAQSKNFHL
RPRDLISNINVIVLELKGSETTFMCEYADETATIVEFLNRWITFCQSIIS
TLT

IL7 is a cytokine that serves as a growth factor for early lymphoid cells of both B- and T-cell lineages. The sequence of IL7 is shown as SEQ ID No. 30.

SEQ ID No. 30
MFHVSFRYIFGLPPLILVLLPVASSDCDIEGKDGKQYESVLMVSIDQLLD
SMKEIGSNCLNNEFNFFKRHICDANKEGMFLFRAARKLRQFLKMNSTGDF
DLHLLKVSEGTTILLNCTGQVKGRKPAALGEAQPTKSLEENKSLKEQKKL
NDLCFLKRLLQEIKTCWNKILMGTKEH

IL15 is a cytokine with structural similarity to IL-2. Like IL-2, IL-15 binds to and signals through a complex composed of IL-2/IL-15 receptor beta chain and the common gamma chain. IL-15 is secreted by mononuclear phagocytes, and some other cells, following infection by virus(es). This cytokine induces cell proliferation of natural killer cells; cells of the innate immune system whose principal role is to kill virally infected cells. The sequence of IL-15 is shown as SEQ ID No. 31.

SEQ ID No. 31
MRISKPHLRSISIQCYLCLLLNSHFLTEAGIHVFILGCFSAGLPKTEANW
VNVISDLKKIEDLIQSMHIDATLYTESDVHPSCKVTAMKCFLLELQVISL
ESGDASIHDTVENLIILANNSLSSNGNVTESGCKECEELEEKNIKEFLQS
FVHIVQMFINTS

The cytokine-based T-cell activating transmembrane protein may comprise one of the following sequences, or a variant thereof:

(membrane-IL7)
SEQ ID No. 32
MAHVSFRYIFGLPPLILVLLPVASSDCDIEGKDGKQYESVLMVSIDQLLD
SMKEIGSNCLNNEFNFFKRHICDANKEGMFLFRAARKLRQFLKMNSTGDF
DLHLLKVSEGTTILLNCTGQVKGRKPAALGEAQPTKSLEENKSLKEQKKL
NDLCFLKRLLQEIKTCWNKILMGTKEHSGGGSPAKPTTTPAPRPPTPAPT
IASQPLSLRPEACRPAAGGAVHTRGLDFACDIYIWAPLAGTCGVLLLSLV
ITLYCNHRNRRRVCKCPRPVV
(membrane-IL15)
SEQ ID No. 33
MGLVRRGARAGPRMPRGWTALCLLSLLPSGFMAGIHVFILGCFSAGLPKT
EANWVNVISDLKKIEDLIQSMHIDATLYTESDVHPSCKVTAMKCFLLELQ
VISLESGDASIHDTVENLIILANNSLSSNGNVTESGCKECEELEEKNIKE
FLQSFVHIVQMFINTSSPAKPTTTPAPRPPTPAPTIASQPLSLRPEACRP
AAGGAVHTRGLDFACDIYIWAPLAGTCGVLLLSLVITLYCNHRNRRRVCK
CPRPVV

The cytokine-based T-cell activating transmembrane protein may comprise a variant of the sequence shown as SEQ ID No. 32 or 33 having at least 80, 85, 90, 95, 98 or 99% sequence identity, provided that the variant sequence is a cytokine-based T-cell activating transmembrane protein having the required properties i.e. the capacity to activate a T cell when present in the envelope protein of a retroviral or lentiviral vector.

### Tagging Protein

The viral envelope of the viral vector may also comprise a tagging protein which comprises a binding domain which binds to a capture moiety and a transmembrane domain.

The tagging protein may comprise:
a binding domain which binds to a capture moiety
a spacer; and
a transmembrane domain.

The tagging protein facilitates purification of the viral vector from cellular supernatant via binding of the tagging protein to the capture moiety.

‘Binding domain’ refers to an entity, for example an epitope, which is capable recognising and specifically binding to a target entity, for example a capture moiety.

The binding domain may comprise one or more epitopes which are capable of specifically binding to a capture moiety. For example the binding domains may comprise at least one, two, three, four or five epitopes capable of specifically binding to a capture moiety. Where the binding domain comprises more than one epitope, each epitope may be separated by a linker sequence, as described herein.

The binding domain may be releasable from the capture moiety upon the addition of an entity which has a higher binding affinity for the capture moiety compared to the binding domain.

### Streptavidin-Binding Epitope

The binding domain may comprise one or more streptavidin-binding epitope(s). For example, the binding domain may comprise at least one, two, three, four or five streptavidin-binding epitopes.

Streptavidin is a 52.8 kDa protein purified from the bacterium Streptomyces avidinii. Streptavidin homo-tetramers have a very high affinity for biotin (vitamin B7 or vitamin H), with a dissociation constant (Kd)˜10⁻¹⁵ M. Streptavidin is well known in the art and is used extensively in molecular biology and bionanotechnology due to the streptavidin-biotin complex's resistance to organic solvents, denaturants, proteolytic enzymes, and extremes of temperature and pH. The strong streptavidin-biotin bond can be used to attach various biomolecules to one another or on to a solid support. Harsh conditions are needed to break the streptavidin-biotin interaction, however, which may denature a protein of interest being purified.

The binding domain may be, for example, a biotin mimic. A ‘biotin mimic’ may refer to an short peptide sequence (for example 6 to 20, 6 to 18, 8 to 18 or 8 to 15 amino acids) which specifically binds to streptavidin.

As described above, the affinity of the biotin/streptavidin interaction is very high. It is therefore an advantage of the present invention that the binding domain may comprise a biotin mimic which has a lower affinity for streptavidin compared to biotin itself.

In particular, the biotin mimic may bind streptavidin with a lower binding affinity than biotin, so that biotin may be used to elute streptavidin-captured retroviral vectors. For example, the biotin mimic may bind streptavidin with a Kd of 1 nM to 100 uM.

The biotin mimic may comprise a sequence as shown in Table 1.

TABLE 1
Biotin mimicking peptides.

| name | Sequence | affinity |
|---|---|---|
| Long nanotag | DVEAWLDERVPLVET | 3.6 nM |
| | (SEQ ID NO: 39) | |
| Short nanotag | DVEAWLGAR | 17 nM |
| | (SEQ ID NO: 40) | |
| Streptag | WRHPQFGG | 72 uM |
| | (SEQ ID NO: 41) | |
| streptagII | WSHPQFEK | |
| | (SEQ ID NO: 36) | |
| SBP-tag | MDEKTTGWRGGHVVEGLAGELEQ | 2.5 nM |
| | LRARLEHHPQGQREP | |
| | (SEQ ID NO: 42) | |
| ccstreptag | CHPQGPPC | 230 nM |
| | (SEQ ID NO: 38) | |
| flankedccstreptag | AECHPQGPPCIEGRK | |
| | (SEQ ID NO: 37) | |

The biotin mimic may be selected from the following group: StreptagII, Flankedccstreptag and ccstreptag.

The binding domain may comprise more than one biotin mimic. For example the binding domain may comprise at least one, two, three, four or five biotin mimics.

Where the binding domain comprises more than one biotin mimic, each mimic may be the same or a different mimic. For example, the binding domain may comprise two StreptagII biotin mimics separated by a linker (for example as shown by SEQ ID NO: 43) or two Flankedccstreptag separated by a linker (for example as shown by SEQ ID NO: 44).

(StreptagII-d8-x2)
SEQ ID NO: 43
WSHPQFEKSGGGGSPAPRPPTPAPTIASWSHPQFEK
(Flankedccstreptag-d8-x2)
SEQ ID NO: 44
ECHPQGPPCIEGRKSSGGGGSPAPRPPTPAPTIASE
CHPQGPPCIEGRKS

### Glutathione S-Transferase

The binding domain may comprise a glutathione S-transferase (GST) domain.

GSTs comprise a family of eukaryotic and prokaryotic phase II metabolic isozymes which catalyze the conjugation of the reduced form of glutathione (GSH) to xenobiotic substrates for the purpose of detoxification. The GST family consists of three superfamilies: the cytosolic, mitochondrial, and microsomal (also known as MAPEG) proteins (Udomsinpraser et al. Biochem. J. (2005) 388 (Pt 3): 763-71).

The GST protein has a strong binding affinity for GSH and this interaction is commonly used in molecular biology to enable the isolation of a GST-tagged protein from a protein mixture.

An amino acid sequence for GST is shown as SEQ ID NO: 45.

(GST)
SEQ ID NO: 45
MGTSLLCWMALCLLGADHADAMSPILGYWKIKGLVQPTRLLLEYLEEKY
EEHLYERDEGDKWRNKKFELGLEFPNLPYYIDGDVKLTQSMAIIRYIAD
KHNMLGGCPKERAEISMLEGAVLDIRYGVSRIAYSKDFETLKVDFLSKL
PEMLKMFEDRLCHKTYLNGDHVTHPDFMLYDALDVVLYMDPMCLDAFPK
LVCFKKRIEAIPQIDKYLKSSKYIAWPLQGWQATFGGGDHPPKSDLEVL
FQGPLG

### Rituximab-Binding Epitope

The present tagging protein may comprise a binding domain which comprises a rituximab-binding epitope (R epitope) and/or a Qbend10 epitope (Q epitope).

A rituximab-binding epitope refers to an epitope which specifically binds rituximab. For example, the rituximab-binding epitope may be based on the CD20 B-cell antigen.

The Rituximab-binding epitope sequence from CD20 is CEPANPSEKNSPSTQYC (SEQ ID No.46)

Perosa et al (2007, J. Immunol 179:7967-7974) describe a series of cysteine-constrained 7-mer cyclic peptides, which bear the antigenic motif recognised by the anti-CD20 mAb Rituximab but have different motif-surrounding amino acids. Eleven peptides were described in all, as shown in the following table:

| Peptide | Insert sequence |
|---|---|
| R15-C | acPYANPSLc (SEQ ID No. 47) |
| R3-C | acPYSNPSLc (SEQ ID No. 48) |
| R7-C | acPFANPSTc (SEQ ID No. 49) |
| R8-, R12-, R18-C | acNFSNPSLc (SEQ ID No. 50) |
| R14-C | acPFSNPSMc (SEQ ID No. 51) |
| R16-C | acSWANPSQc (SEQ ID No. 52) |
| R17-C | acMFSNPSLc (SEQ ID No. 53) |
| R19-C | acPFANPSMc (SEQ ID No. 54) |
| R2-C | acWASNPSLc (SEQ ID No. 55) |
| R10-C | acEHSNPSLc (SEQ ID No. 56) |
| R13-C | acWAANPSMc (SEQ ID No. 57) |

Li et al (2006 Cell Immunol 239:136-43) also describe mimetopes of Rituximab, including the sequence:

(SEQ ID No. 58)
QDKLTQWPKWLE.

The polypeptide of the present invention comprises a Rituximab-binding epitope having an amino acid sequence selected from the group consisting of SEQ ID No. 46-58 or a variant thereof which retains Rituximab-binding activity.

### QBend10

The CliniMACS CD34 selection system utilises the QBEnd10 monoclonal antibody to achieve cellular selection. The present inventors have previously mapped the QBEnd10-binding epitope from within the CD34 antigen (see WO 2013/153391) and determined it to have the amino acid sequence shown as SEQ ID No. 59.

(SEQ ID No. 59)
ELPTQGTFSNVSTNVS.

The binding domain of the present tagging protein the present invention may comprise a QBEnd10-binding epitope having the amino acid sequence shown as SEQ ID No. 59 or a variant thereof which retains QBEnd10-binding activity.

### RQR8

The tagging protein may comprise a binding domain which comprises or consists of 136 amino acid sequence shown as SEQ ID NO: 60.

(RQR8)
SEQ ID NO: 60
CPYSNPSLCSGGGGSELPTQGTFSNVSTNVSPAKPTTTACPYSNPSLCSG
GGGSPAPRPPTPAPTIASQPLSLRPEACRPAAGGAVHTRGLDFACDIYIW
APLAGTCGVLLLSLVITLYCNHRNRRRVCKCPRPVV

### Nucleic Acid

The invention also relates to a nucleic acid encoding a cytokine-based T-cell activating transmembrane protein or a nucleic acid encoding a mitogenic T-cell activating transmembrane protein. The nucleic acid may be in the form of a construct comprising a plurality of sequences encoding a mitogenic T-cell activating transmembrane protein and/or a cytokine-based T-cell activating transmembrane protein.

As used herein, the terms “polynucleotide”, “nucleotide”, and “nucleic acid” are intended to be synonymous with each other.

It will be understood by a skilled person that numerous different polynucleotides and nucleic acids can encode the same polypeptide as a result of the degeneracy of the genetic code. In addition, it is to be understood that skilled persons may, using routine techniques, make nucleotide substitutions that do not affect the polypeptide sequence encoded by the polynucleotides described here to reflect the codon usage of any particular host organism in which the polypeptides are to be expressed.

Nucleic acids may comprise DNA or RNA. They may be single-stranded or double-stranded. They may also be polynucleotides which include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of the use as described herein, it is to be understood that the polynucleotides may be modified by any method available in the art. Such modifications may be carried out in order to enhance the in vivo activity or life span of polynucleotides of interest.

The terms “variant”, “homologue” or “derivative” in relation to a nucleotide sequence include any substitution of, variation of, modification of, replacement of, deletion of or addition of one (or more) nucleic acid from or to the sequence.

The nucleic acid may produce a polypeptide which comprises one or more sequences encoding a mitogenic T-cell activating transmembrane protein and/or one or more sequences encoding a cytokine-based T-cell activating transmembrane protein. The cleavage site may be self-cleaving, such that when the polypeptide is produced, it is immediately cleaved into the receptor component and the signalling component without the need for any external cleavage activity.

Various self-cleaving sites are known, including the Foot-and-Mouth disease virus (FMDV) 2a self-cleaving peptide and various variants and 2A-like peptides. The peptide may have the sequence shown as SEQ ID No. 34 or 35.

SEQ ID No. 34
RAEGRGSLLTCGDVEENPGP.
SEQ ID No 35
QCTNYALLKLAGDVESNPGP

The co-expressing sequence may be an internal ribosome entry sequence (IRES). The co-expressing sequence may be an internal promoter.

### Vector

The present invention also provides a vector, or kit of vectors which comprises one or more sequences encoding a mitogenic T-cell activating transmembrane protein and/or one or more sequences encoding a cytokine-based T-cell activating transmembrane protein. Such a vector may be used to introduce the nucleic acid sequence(s) into a host cell, such as a producer or packaging cell.

The vector may, for example, be a plasmid or a viral vector, such as a retroviral vector or a lentiviral vector, or a transposon based vector or synthetic mRNA.

The vector may be capable of transfecting or transducing a host cell.

### Method

The invention also provides a method for making an activated transgenic T-cell or natural killer (NK) cell, which comprises the step of transducing a T or NK cell with a retroviral or lentiviral vector according to the invention, such that the T-cell or NK cell is activated by one or more mitogenic T-cell activating transmembrane protein(s) and optionally one or more cytokine-based T-cell activating transmembrane protein(s).

The method for transducing and activating T cells or NK calls may take 48 hours or less, for example between 24 and 48 hours.

The invention will now be further described by way of Examples, which are meant to serve to assist one of ordinary skill in the art in carrying out the invention and are not intended in any way to limit the scope of the invention.

### Examples

### Example 1—Production of Viral Vectors Displaying OKT3 on the Virion Surface

An initial proof-of-concept experiment was performed where it was demonstrated that expression of an OKT3 scFv on the packaging cell results the production of viral vector which causes the mitogenic activation of T-cell targets.

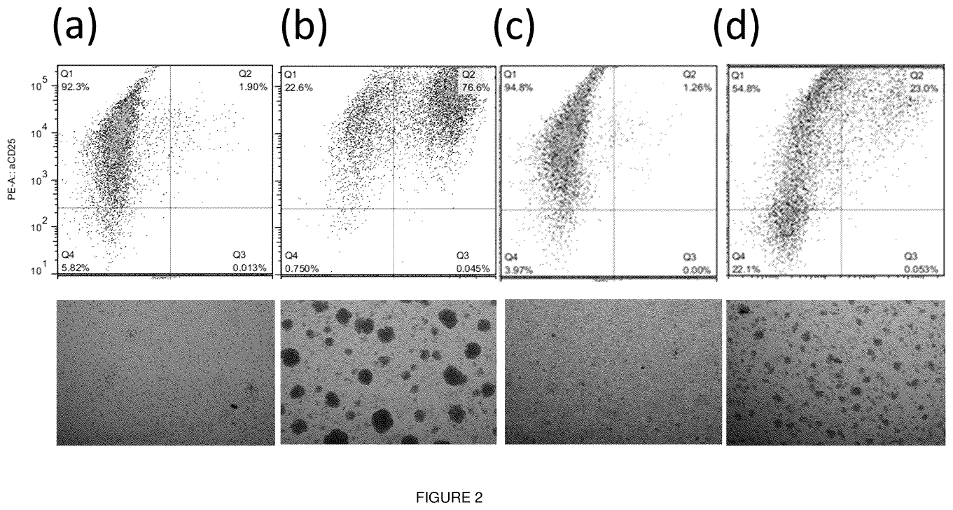

OKT3 scFv 293T cells produced a lentiviral vector which caused activation and transduction of target T-cells. This mitogenic property was dependent on the presence of lentiviral helper components i.e. the effect was not due to a non-specific property of the packaging cell supernatant (FIG. 2).

A comparison of the OKT3 scFv attached to the membrane via a CD8 stalk or via an IgG1 hinge's ability to incorporate into the lentivirus and cause a mitogenic stimulus was also made, with no difference noted between the two spacers.

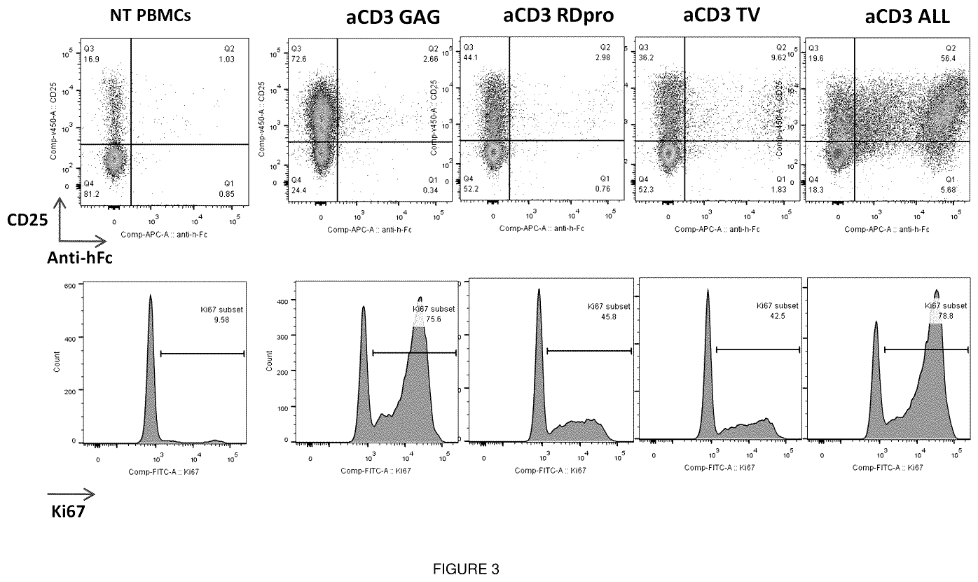

293T cells stably expressing surface bound OKT3 were transfected with lentiviral gagpol, RD-PRO env, the transfer vector or all three plasmids along with a lentiviral rev expressing plasmid. The subsequent supernatant was applied to primary human T-cells. The T-cells were subsequently studied by flow-cytometry with the following parameters: CD25 to measure T-cell activation; anti-Fc to detect transgene (CAR with an Fc space) r; ki67 to determine cells in cycle (FIG. 3). Only conditions where gagpol was supplied resulted in significant mitogenic stimulation.

Only the condition where all plasmids were supplied (along with rev) resulted in mitogenic stimulation of T-cells and transduction

Further experiments were also conducted to determine if different lentiviral pseudotyping supports the mitogenic effect. 293T cells stably expressing the membrane bound OKT3 were transfected with a lentiviral transfer vector, lentiviral gagpol, rev and different env plasmids: namely VSV-G, RD-PRO, Ampho, GALV and

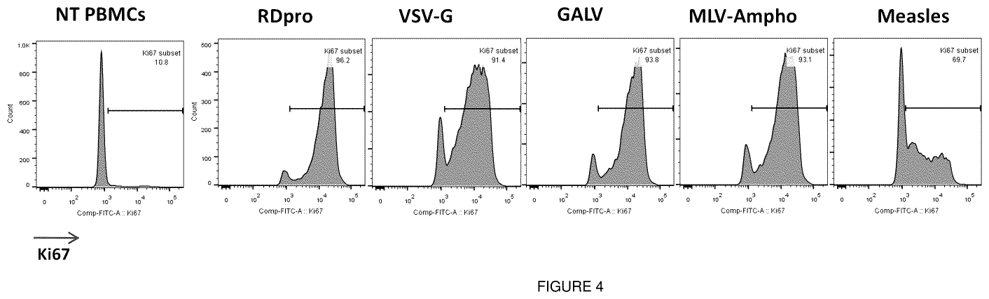

Measles M/H. The subsequent supernatant was applied to primary human T-cells. The cells were subsequently stained with ki67 and studied by flow-cytometry. All pseudotypes supported the mitogenic effect, although the effect seemed reduced with Measles pseudotyping (FIG. 4).

### Example 2—Two Separate Mitogenic Stimuli can be Incorporated into the Viral Vector

An additional construct which comprised the anti-CD28 activating scFv from antibody 15E8 was generated. The OKT3 scFv cassette (described above) expressed eGFP and the 15E8 scFv cassette expressed the blue fluorescent protein eBFP2.

293T cells were generated which co-expressed high levels of eGFP and eBFP2, demonstrating the successful expression of both OKT3 and 15E8 on the surface of the 293T cells.

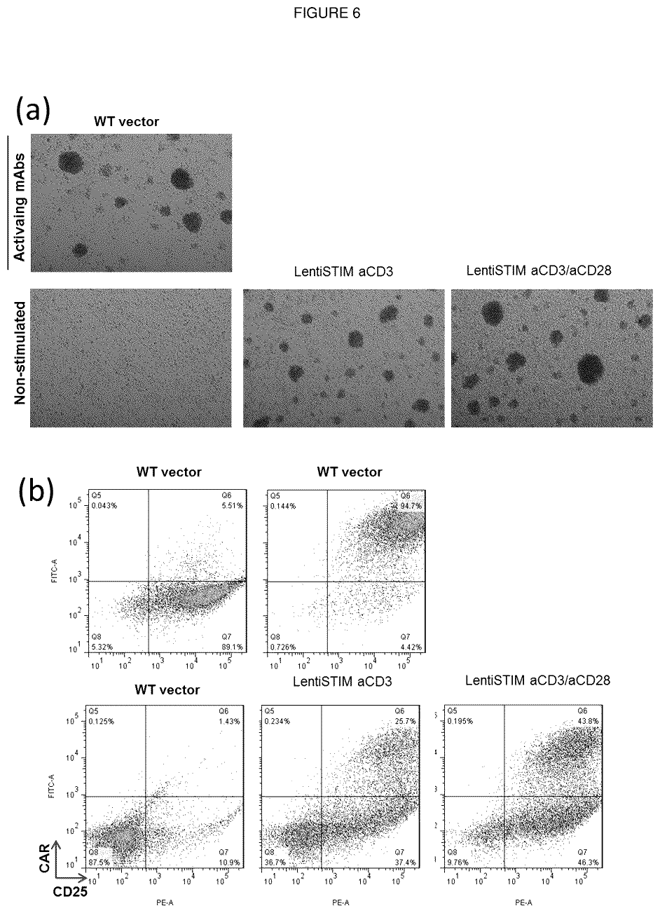

Lentiviral supernatant was generated from wild-type 293T cells, 293T cells which expressed OKT3 scFv alone and 293T cells which expressed both OKT3 and 15E8. Activation levels and transduction efficiency was greater with the two stimulations (FIG. 6).

### Example 3—Demonstration of Functionality in Gamma-Retroviral Vectors

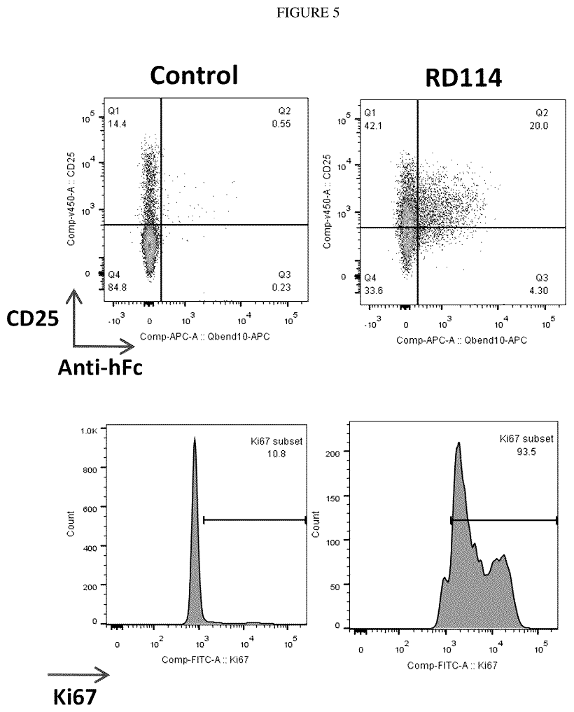

293T cells stably expressing membrane bound OKT3 were transfected with a gamma-retroviral transfer vector coding for a CAR, gamma-retroviral gagpol expression plasmid and an RD114 expression plasmid. Subsequent supernant was applied to primary human T-cells. The T-cells were stained with anti-Fc, anti-CD25 and ki67 and studied by flow-cytometry. Although no mitogenic stimulus was applied, T-cells were activated, cycling and were expressing transgene (FIG. 5)

### Example 4—Combinations of Peptides Incorporated into Lentivirus Vectors

Different combinations of elements were incorporated into packaging cell lines. This included TGN1412 scFv which is a super-agonistic anti-CD28 mAb. Cytokines IL7 and IL15, as well as OX40L and 41BBL were also incorporated in different combinations as follows:
1. (Nil)
2. OKT3
3. OKT3+15E8
4. OKT3+TGN1412
5. OKT3+15E8+OX40L+41BBL
6. OKT3+15E8+OX40L+41BBL+mIL15
7. OKT3+15E8+OX40L+41BBL+mIL7

Lentiviral vector generated from these different 293T cells was used to stimulate/transduce T-cells.

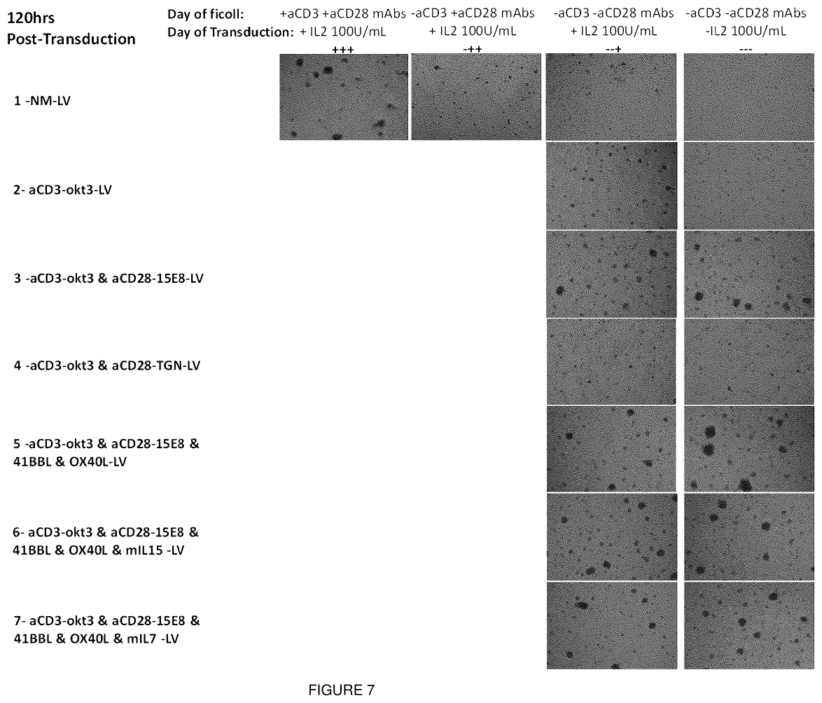

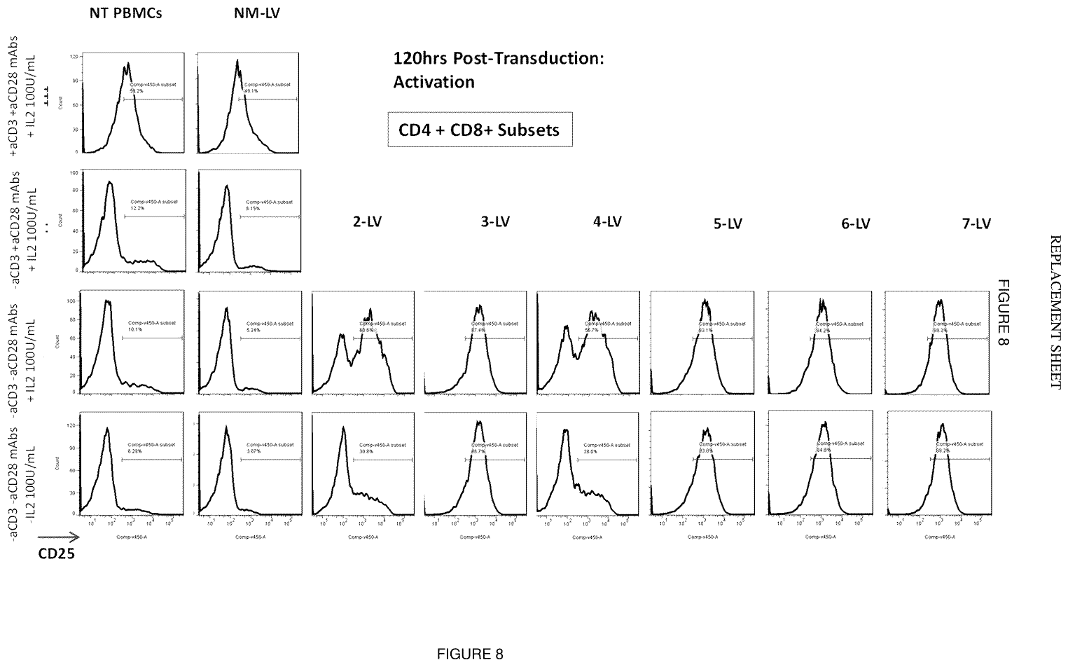

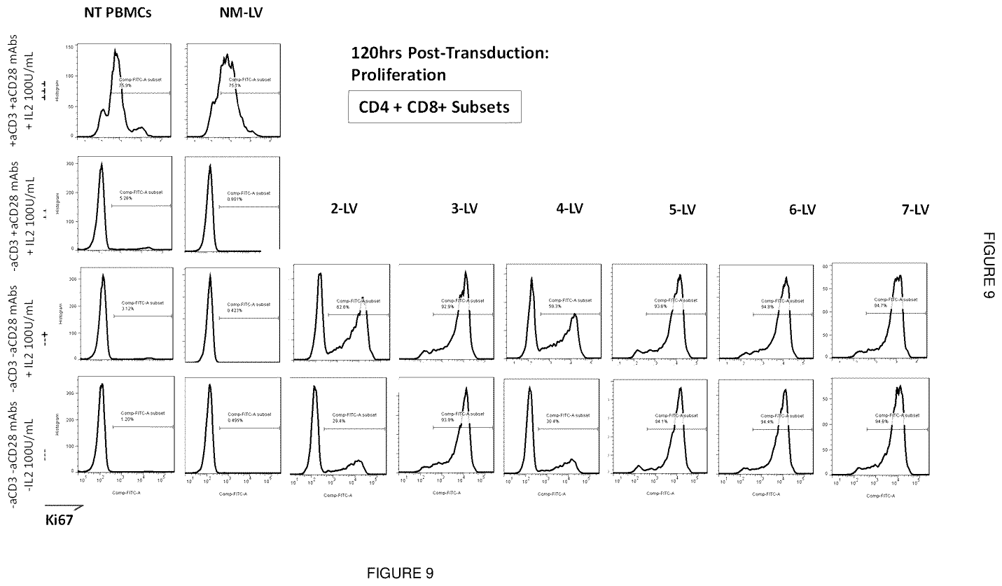

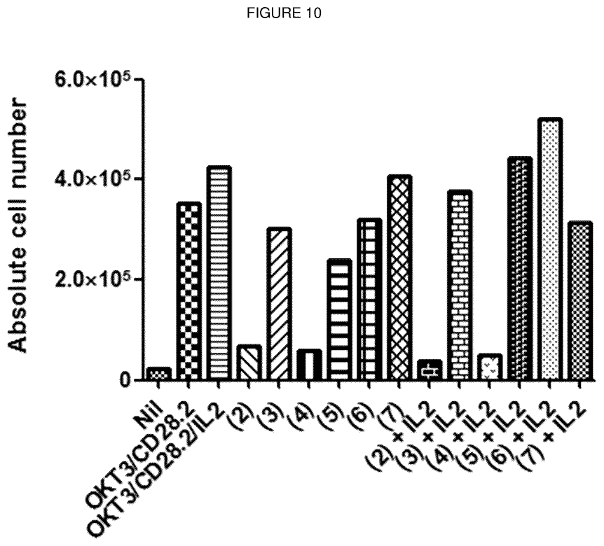

Vector generated from non-engineered 293T cells along with mitogenic soluble antibodies OKT3 and CD28.2+/−IL2 was used as a control. Activation (CD25), proliferative fraction (Ki67) and absolute counts at day 5 were measured (FIGS. 7-10).

It was once again noted that there was a marked advantage of incorporating two signals instead of one. It was also noted that activation using mitogenic peptides displayed on the virion surface was markedly superior to the activation achieved by adding soluble antibodies to the T-cells.

Similar levels of proliferation to that of mAb activation with cytokine were also achieved.

### Methodology

The VH and VL of mitogenic antibodies were cloned as scFvs and connected to a spacer domain, a TM domain and a polar anchor (SEQ ID Nos 1-3 above)

Cytokines were connected in frame to a spacer, a TM domain and a polar anchor (SEQ ID Nos 32 and 33 above).

For native co-stimulatory molecules such as 41BBL and OX40L, these are cloned in their native forms.

Each of the above types of membrane-bound proteins could then be stably expressed at high-levels on a 293T cell.

Viral vectors were made from these 293T cells using standard transient transfection. For lentiviral vector the transfer vector, rev expression vector, a lenti gagpol expression vector and the RD-PRO expression vector were co-transfected. For gamma retroviral vectors, the 293T cells were co-transfected with the transfer vector, MoMLV gagpol and RD114 expression plasmid. The supernatant was clarified by centrifugation and filtration with a 0.45 uM filter. The virus was applied to primary human T-cells on a retronectin plate. IL2 is added in some conditions, or no cytokines are added in other conditions.

### Example 5—Comparing T Cell Subset Phenotypes from Cells Stimulated with Lentiviral Vector Versus Cells Stimulated with AntiCD3/AntiCD28 Antibody-Coated Beads

Mononuclear cells were isolated from peripheral blood using standard techniques. Peripheral blood mononuclear cells (PBMCs) were then treated with either:
(i) antiCD3/antiCD28 antibody-coated beads (Dynabeads® Human T-Activator CD3/CD28) in a 3:1 ratio in the presence of non-modified lentiviral vector and IL15/IL7; or
(ii) lentiviral vector expressing OKT3 and 15E8B (combination 3 as described in Example 4) on a retronectin-coated plate in the presence of IL15 and IL7.

After 48 hours, cells were harvested and stained with a panel of T-cell phenotyping antibodies, as follows:
aCD4-BV650
aCD8-PE.Cy7
aCD45RO-BV605
aCD45RA-FITC
aCD95-PB
aCD197-BV685

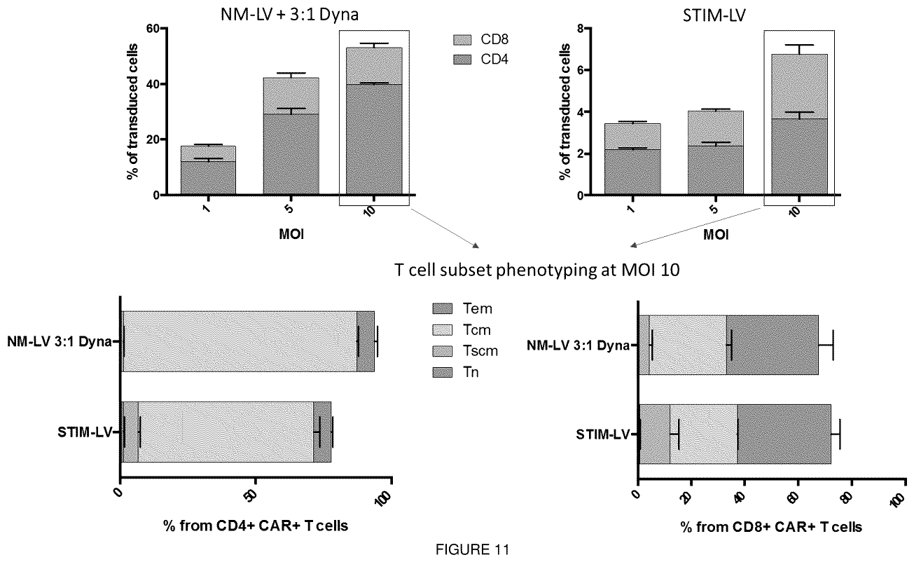

T cell subsets were analysed by FACS and the results are summarised in FIG. 11. For both the CD4+ and CD8+ T cell subsets, the cells stimulated with virus showed a greater proportion of naïve T cells (Tn and Tscm) than cells stimulated with antibody-coated beads.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology, cellular immunology or related fields are intended to be within the scope of the following claims.

1.-26. (canceled)

27. A kit for making a retroviral or lentiviral vector having a viral envelope that comprises a first mitogenic T-cell activating transmembrane protein which comprises a mitogenic domain which binds CD3 and comprises a transmembrane domain, and a second mitogenic T-cell activating transmembrane protein which comprises a mitogenic domain which binds CD28 and comprises a transmembrane domain, the kit comprising:
(a) a host cell which expresses at the cell surface, the first and second mitogenic T-cell activating transmembrane proteins, wherein the mitogenic T-cell activating transmembrane proteins are not part of a viral envelope glycoprotein,
(b) nucleic acids comprising gag, pol, env and optionally rev, and
(c) a retroviral or lentiviral genome.

28. A kit for making a packaging cell which comprises:
(a) one or more nucleic acid(s) encoding:
(i) a first mitogenic T-cell activating transmembrane protein which comprises a mitogenic domain which binds CD3 and comprises a transmembrane domain and
(ii) a second mitogenic T-cell activating transmembrane protein which comprises a mitogenic domain which binds CD28 and comprises a transmembrane domain, wherein the mitogenic T-cell activating transmembrane proteins are not part of a viral envelope glycoprotein, and
(b) nucleic acids comprising retroviral gag, pol, env and optionally rev genes.

29. A kit for making a producer cell which comprises:
(a) one or more nucleic acid(s) encoding:
(i) a first mitogenic T-cell activating transmembrane protein which comprises a mitogenic domain which binds CD3 and comprises a transmembrane domain and
(ii) a second mitogenic T-cell activating transmembrane protein which comprises a mitogenic domain which binds CD28 and comprises a transmembrane domain, wherein the mitogenic T-cell activating transmembrane proteins are not part of a viral envelope glycoprotein,
(b) nucleic acids comprising retroviral gag, pol, env and optionally rev genes, and
(c) a retroviral or lentiviral vector genome.
